(19) United States
(12) Patent Application Publication
(10) Pub. No.: US 2018/0132911 A1
(43) Pub. Date: May 17, 2018

(54) GRASPING END OF FROG-STYLE FORCEPS

(71) Applicant: SHANDONG WEIGAO ORTHOPEDIC DEVICE COMPANY LTD, Weihai (CN)

(72) Inventors: Chunhui WU, Weihai (CN); Haibo ZOU, Weihai (CN); Xiujun ZHENG, Weihai (CN); Yajun GAO, Weihai (CN); Chaoliang YU, Weihai (CN); Haichen CONG, Weihai (CN)

(21) Appl. No.: 15/571,851

(22) PCT Filed: May 5, 2016
(86) PCT No.: PCT/CN2016/081098

§ 371 Date: Nov. 6, 2017

(30) Foreign Application Priority Data
May 12, 2015 (CN) 201510238712.1

(51) Int. Cl. A61B 17/70; A61B 17/28

(52) U.S. Cl. CPC A61B 17/7088; A61B 17/282

(57) ABSTRACT

A grasping end of a frog-style forceps relates to the technical field of medical instrument for spinal surgery, and comprises a grasping end tube and a pressing-rod sleeve. The pressing-rod sleeve is sleeved over the grasping end tube. Two opposite strip-shaped openings are axially provided on a side wall of the grasping end tube. Positioning spring sheets are disposed inside the strip-shaped openings. Positioning protrusions are disposed on inner sides of lower end portions of the two positioning spring sheets inside the strip-shaped openings at a lower portion of the grasping end tube. A position limiting protrusion is disposed inside the lower portion of the grasping end tube. The grasping end for a frog-style forceps does not need to align relatively small positioning protrusions with a slot on a screw seat, so that it is convenient and easy to assemble and very easy to dismantle the grasping end.

### TECHNICAL FIELD

The present disclosure relates to the technical field of medical instrument for spinal surgery. More particularly, it relates to a grasping end for frog-style forceps that is convenient, simple, and highly efficient to assemble, very easy to dismantle, and both time- and labor-saving during the rod pressing process.

### BACKGROUND

As we all know, the rod pressing is an intraoperative operation that is often required for spine orthopedics and posterior spine minimally invasive surgery, the method of which is to push a connecting rod into the U-shaped slot of a screw by means of tools or long-tailed pedicle screw. For multi-segment posterior thoracolumbar surgery, in most cases, when a pedicle screw is implanted into the patient's vertebral body, the screw seat positions of the pedicle screw cannot be in a straight line or fully align with the axis of the fixed rod. As a result, the connecting rod cannot be inserted into the remaining screw seats easily after the connecting rod has been placed into some of the screw seats, so that it is necessary to press the rod into the remaining screw seats. The frog-style forceps of the Legacy screw-rod system from Medtronic Company has been widely used in the rod pressing operation. The State Intellectual Property Office of China published a utility model patent No. 201220212690.3 entitled “Frog-Style Forceps” filed on May 11, 2012, which discloses the basic structure of frog-style forceps in the prior art. The existing grasping end of frog-style forceps comprises a grasping end tube and a rod pressing sleeve arranged on the outer side of the grasping end tube, both adapted to cooperate and couple with the screw seat. The side wall of the grasping end tube is provided axially with two opposing open grooves that each have an open lower end and divide the lower side of the grasping end tube into two symmetrical half-tubular clamping pieces. The lower ends of the two clamping pieces slightly expand outward, and are provided with, on their inner side, a positioning protrusion cooperating with the groove on the side wall of the screw seat and also provided with a U-shaped slot cooperating with the connecting rod. In use, the front end of the grasping end tube is first aligned with the screw seat and placed downward onto an appropriate height to the screw seat, so that the positioning protrusion of the grasping end tube is aligned with the groove of the upper side of the screw. Then the forceps' handle is engaged to push the rod pressing sleeve to move downward, and two clamping pieces on the front end of the grasping end tube are compressed to fold, so that the positioning protrusion of the clamping pieces enters into the groove of the side wall of the screw seat.

### SUMMARY

### Technical Problem

Since it is difficult to observe the relative position of the positioning protrusion and the groove of the side wall of the screw seat during the operation, the positioning projection is often stuck outside the groove of the screw seat, so that the rod pressing sleeve cannot move downward to implement the rod pressing action or may get loose during the rod pressing process. In addition, after the rod pressing is finished, the frog-style forceps often can only be removed by repeatedly shaking the grasping end tube or sometimes even cannot be removed, in which case the entire screw has to be removed, which seriously hinders the successful completion of the operation. Due to the large force required to compress the two clamping pieces of the lower side of the grasping end tube inward, a large frictional force is generated during the downward movement of the rod pressing sleeve, causing easy fatigue of the doctor's hand during the operation.

### Technical Solution

One object of the present disclosure aims to solve the above-mentioned deficiencies of the prior art and provides a grasping end of frog-style forceps that is convenient, simple, and highly efficient to assemble, very easy to dismantle, and both time- and labor-saving during the rod pressing process.

The technical solution of the present disclosure to solve the above-mentioned deficiencies of the prior art is that:

A grasping end for frog-style forceps, comprising a grasping end tube and a rod pressing sleeve that is provided on the outer side of the grasping end tube, wherein two opposing elongated openings are axially arranged on the side wall of the grasping end tube, with a positioning spring piece provided inside each elongated opening. A positioning protrusion is provided on the inner side of the lower end of each of the two positioning spring pieces inside the elongated openings of the lower part of the grasping end tube. A limiting protrusion is provided inside the lower part of the grasping end tube. When the rod pressing sleeve is placed on the upper side of the grasping end tube and not pressed against the positioning spring piece yet, the lower ends of both positioning spring pieces expand outward, wherein the maximum distance between the outer side walls of the two positioning spring pieces is greater than the inner diameter of the rod pressing sleeve, and the minimum distance between the two positioning protrusions is not less than the diameter of the screw seat. When the rod pressing sleeve slides downward to the underside of the grasping end tube and is pressed against the positioning spring pieces, the minimum distance between the two positioning protrusions is smaller than the diameter of the screw seat. When the limiting protrusion is in contact with the upper end surface of the screw seat, the axial heights of the two positioning protrusions are the same as the axial height of the groove on the outer side wall of the screw seat.

The positioning spring piece according to the present disclosure has an elongated strip shape, an upper end of which is connected to the grasping end tube at an upper part of the elongated opening.

The lower end of the grasping end tube according to the present disclosure is provided with two opposing U-shaped slots which cooperate with the connecting rod. The angle between the U-shaped slot and the adjacent elongated opening is 90°.

The upper ends of the grasping end tube and the rod pressing sleeve according to the present disclosure are provided with connecting seats adapted to connect the body of the frog-style forceps, respectively.

The outer side of the lower end of the positioning spring piece according to the present disclosure is provided with a pressing slope which cooperates with the lower end surface of the rod pressing sleeve. When the rod pressing sleeve slides downward, its lower end surface cooperates with the pressing slope, so that the positioning spring piece can be pressed inward more easily.

The lower end of the rod pressing sleeve according to the present disclosure is provided with an arc-shaped groove cooperating with the connecting rod. During rod pressing rod, the arc-shaped groove is in contact with the connecting rod surface, so that the operation is more stable.

### Advantageous Effects

The present disclosure does not require alignment of the relatively small positioning protrusions to the grooves of the screw seat. Therefore, it is convenient and labor-saving to assemble and very easy to dismantle. Repeatedly aligning and engaging the handle during the screw seat connecting is completely avoided. The friction force during the rod pressing process is small. It is also easy to remove the frog-style forceps coupled with the screw seat without repeated shaking. The operation is thus time- and labor-saving, which reduces the operation time greatly.

### BRIEF DESCRIPTION OF THE DRAWINGS

FIG. 1 is a schematic structural view of the present disclosure.

FIG. 2 is a cross-sectional view taken along the line A-A of FIG. 1.

FIG. 3 is a schematic structural view of the present disclosure coupled with the screw seat of the pedicle screw.

FIG. 4 is a schematic 3D-structural view of he present disclosure coupled with the screw seat of the pedicle screw.

FIG. 5 is a schematic structural view of the frog-style forceps according to the present disclosure.

### DETAILED DESCRIPTION

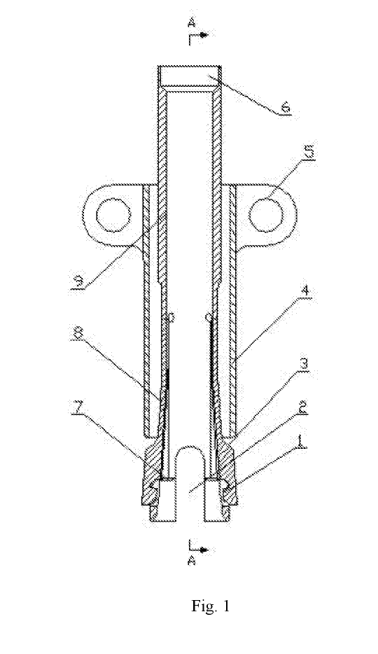

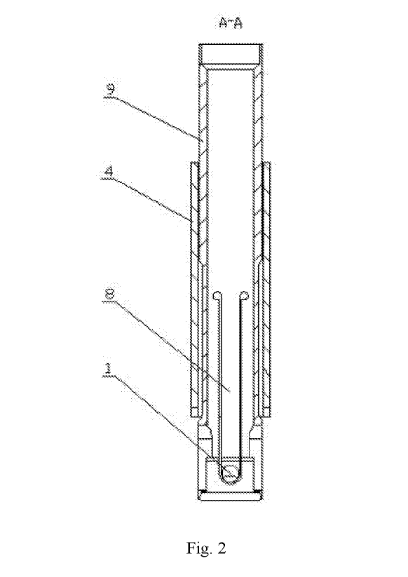

As shown in FIGS. 1 and 2, a grasping end of frog-style forceps comprises a grasping end tube 9 and a rod pressing sleeve 4 that is provided on the outer side of the grasping end tube 9. Two opposing elongated openings are axially arranged and radially penetrate the side wall of the grasping end tube 9. A positioning spring piece 8 is provided inside elongated openings, and as can be seen from the figure, the positioning spring piece 8 has an elongated strip shape. An upper end of the positioning spring piece 8 is connected to the grasping end tube at an upper part of the elongated opening. The lower end of the grasping end tube is provided with two opposing U-shaped slot 2 which cooperate with the connecting rod. The angle between the U-shaped slot 2 and the adjacent elongated opening is 90°. The opposite inner sides of the lower ends of the two positioning spring piece 8 provided inside the elongated opening of the upper part of the grasping end tube 9 is provided with a positioning protrusion 1 cooperating with the groove on the side wall of the screw seat. The lower part of the grasping end tube is provided with a limiting protrusion 7 for blocking the screw seat. When the rod pressing sleeve is placed on the upper side of the grasping end tube and not pressed against the positioning spring piece yet, the lower ends of both positioning spring pieces expand outward, wherein the maximum distance between the outer side walls of the two positioning spring pieces is greater than the inner diameter of the rod pressing sleeve, and the minimum distance between the two positioning protrusion is not less than the diameter of the screw seat. When the rod pressing sleeve slides downward to the underside of the grasping end tube and is pressed against the positioning spring pieces, the minimum distance between the two positioning protrusions is smaller than the diameter of the screw seat. When the limiting protrusion is in contact with the upper end surface of the screw seat, the axial heights of the two positioning protrusion are the same as the axial height of the groove provided on the outer side wall of the screw seat. The upper ends of the grasping end tube and the rod pressing sleeve are provided with connecting seats 6 and 5, which are adapted to connect to a forcep body of the frog-style forceps.

The present disclosure can be further modified by providing a pressing slope 3 on the outer side of the lower end of the positioning spring piece, for cooperating with the lower end surface of the rod pressing sleeve. When the rod pressing sleeve slides downward, its lower end surface cooperates with the pressing slope 3, so that the positioning spring piece can be pressed inward more easily. The lower end of the rod pressing sleeve is provided with an arc-shaped groove which cooperating with the connecting rod. During rod pressing, the arc-shaped groove is in contact with the connecting rod surface, so that the operation is more stable.

### Industrial Utility

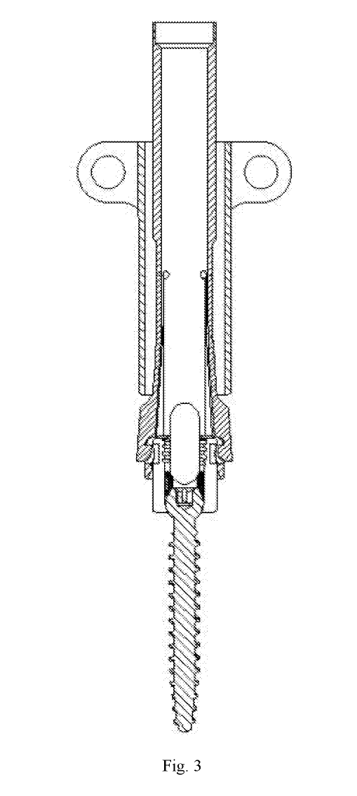

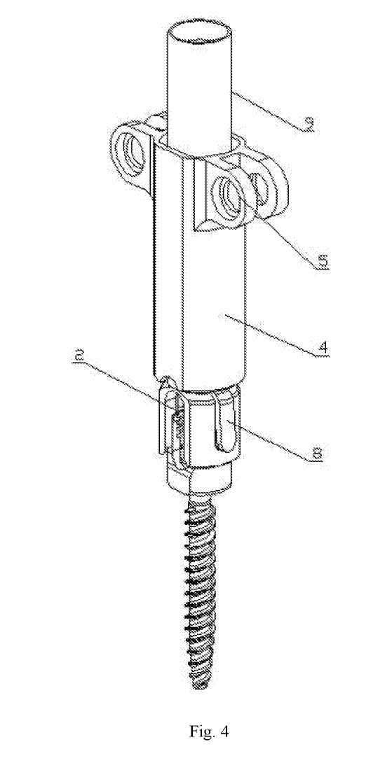

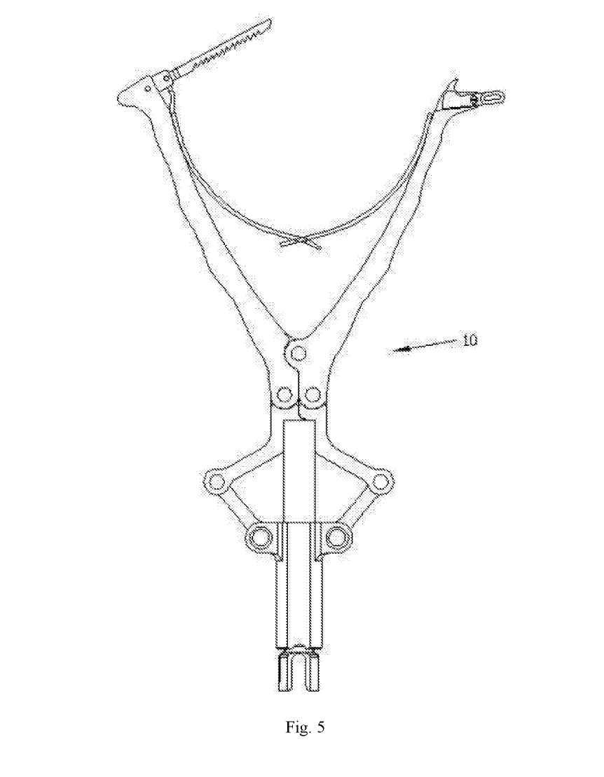

As shown in FIGS. 3 and 4, when the present disclosure is in use, first the lower end of the grasping end tube is aligned with and then slide down into the screw seat, and the U-shaped slot is stuck on the connecting rod. When the limiting protrusion meets with the upper surface of the side wall of the screw seat, the positioning protrusion on the positioning spring piece is aligned with the groove on the side walls of the screw seats automatically. At this point, the handle of the frog-style forceps can be folded, so that the rod pressing sleeve moves downward. First, the edge of lower opening of the rod pressing sleeve touches the outer side of the positioning spring pieces that spontaneously expand, which causes the positioning spring pieces to fold inward, and the positioning protrusions on the positioning spring pieces enter into the grooves on both sides of the screw seat wall, so that the grasping end tube is connected to the screw seat tightly without loosening. As the handle continues to fold, the rod pressing sleeve continues to move downward, and then the rod pressing sleeve will meet the connecting rod on the screw seat and press it down to the bottom of the screw seat. Due to the good elasticity of the positioning spring pieces, the reactive force on the rod pressing sleeve is small, so that the friction is also small. When the rod pressing action is completed, other surgical operations are often needed to implement, such as screwing the plug screw onto the screw seat through the longitudinal center hole of the grasping end tube, as a substitute of the rod pressing sleeve, to lock the connecting rod with the screw seat. When all operations are completed, the handle can be loosen, and the rod pressing sleeve moves upward. The positioning spring pieces expand outward under their own elasticity, and the positioning protrusions on the positioning spring pieces separate from the groove on the side wall of the screw seat, so that the grasping end tube can be removed from the screw seat directly without shaking repeatedly. Therefore, the reliability of the rod pressing device is improved greatly. As shown in FIG. 5, the present disclosure can be connected to the body 10 of the frog-style forceps to form frog-style forceps.

The present disclosure does not require alignment of the relatively small positioning protrusions to the grooves of the screw seat. Therefore, it is convenient and labor-saving to assemble and very easy to dismantle. Repeatedly aligning and engaging the handle during the screw seat connecting is completely avoided. The friction force during the rod pressing process is small. It is also easy to remove the frog-style forceps coupled with the screw seat without repeated shaking. The operation is thus time- and labor-saving, which reduces the operation time greatly.

1. A grasping end for frog-style forceps, comprising a grasping end tube and a rod pressing sleeve that is sleeved on an outer side of the grasping end tube, wherein two opposing elongated openings are axially arranged in a side wall of the grasping end tube, two positioning spring pieces are provided in the two elongated openings respectively;
a positioning protrusion is provided on an inner side of a lower end of each of the two positioning spring pieces provided in the elongated openings at a lower part of the grasping end tube; and,
a limiting protrusion is provided inside the lower part of the grasping end tube.

2. The grasping end for frog-style forceps according to claim 1, wherein the positioning spring piece has an elongated strip shape, and an upper end of the positioning spring piece is connected to the grasping end tube at an upper part of the elongated opening.

3. The grasping end for frog-style forceps according to claim 1, wherein an lower end of the grasping end tube is provided with two opposing U-shaped slots which cooperate with a connecting rod.

4. The grasping end for frog-style forceps according to claim 3, wherein each of an upper end of the grasping end tube and an upper end of the rod pressing sleeve is provided with a connecting seat adapted to be connected to a forcep body of the frog-style forceps.

5. The grasping end for frog-style forceps according to claim 4, wherein an outer side of the lower end of the positioning spring piece is provided with a pressing slope which cooperates with a surface of the lower end of the rod pressing sleeve.

6. The grasping end for frog-style forceps according to claim 4, wherein an lower end of the rod pressing sleeve is provided with an arc-shaped groove which cooperates with the connecting rod.
